(19) United States
(12) Patent Application Publication
(10) Pub. No.: US 2006/0167421 A1
(43) Pub. Date: Jul. 27, 2006

(54) Catheter with insert-molded tip

(75) Inventor: David G. Quinn, Grayslake, IL (US)

Correspondence Address: BRINKS HOFER GILSON & LIONE, P.O. BOX 10395, CHICAGO, IL, 60610, US

(73) Assignee: Radius International Ltd. Partnership

(21) Appl. No.: 11/336,339

(22) Filed: Jan. 20, 2006

Related U.S. Application Data
(60) Provisional application No. 60/646,215, filed on Jan. 21, 2005.

(51) Int. Cl. A61M 5/32

(52) U.S. Cl. 604/274

(57) ABSTRACT

A single lumen catheter includes a catheter tube and an overmolded tip. The catheter is fabricated by skiving a portion of the distal end of a tube. The distal end is then inserted in a mold and molten polyurethane or silicone, for example, is injected into the mold. The configuration of the tip and its relation to the tube creates an impediment to kinking of the tip or the tube where it joins the tip.

### RELATED APPLICATION

This application is based on Provisional application Ser. No. 60/646,215 and claims priority therefrom. The disclosure of the Provisional application is incorporated herein in its entirety by reference.

### FIELD OF THE INVENTION

This invention relates generally to a method of manufacturing a single lumen catheter tip for any number of medical uses including intravenous access, urology access and enteral access. Catheter shape and function has previously been described in Quinn U.S. Pat. No. 5,451,490 and No. 5,599,322. The invention relates specifically to a method of overmolding/insert molding a tip that has the same OD as the tube and incorporates an arch or dimple in its preferred embodiment that reinforces the port section of the tube to prevent kinking.

### BACKGROUND OF THE INVENTION

The existing tips of single lumen catheters for medical purposes have either open ended ports cut at 90 degrees to the longitudinal length of the tube or rounded bullet tips. The square cut tubes are damaging to vessel walls during insertion and are prone to occlusion during the aspiration mode. In situ these square edges continually scratch the vessel wall causing the build up of fibrin sheaths that ultimately block flow and act as clots if they break loose from the tip. Most cut off tubes also have side hole ports that are smaller than the ID of the tube to prevent kinking.

The round bullet shape tubes are formed di-electrically or with heat by pushing a square cut tube into a female mold that forms the tip, or by gluing a pre-molded bullet shaped cap into the lumen of the tube. Ports are then punched into the tube along its length. Bullet tips that have OD's larger than the tube itself are formed in the manner of pre-mentioned Quinn patents. The Quinn inventions allow for larger effective ports, better aspiration, easier safer insertion, less occlusion and an improved, softer edge insitu.

The C. R. Bard Groshong percuteneously inserted cardiac catheter (PIIC) has a bullet tip formed in silicone on a silicone tube. The tip of the Groshong has a longitudinal slit that acts as a valve and port. The disadvantages of this slit is that it hinders flow because it must be forced open and the exit velocity of the infusate from the slit is therefore accelerated. This outflow is directly forced against the vessel wall in a very forceful stream.

The infusates commonly utilized in PIIC catheters are very caustic and are infused at high rates of flow with hand held syringes thereby irritating the vessel wall at the point of contact with the infusate.

The latest Quinn invention slows the exist velocity and diffuses the infusate through its larger port around the over-molded tip. Another disadvantage of the Groshong tip/slit valve is that it must be constructed of silicone because the slit valve will not function with the stiffer polyurethane. Urethane is much stronger than Silicone and is less likely to break insitu.

### SUMMARY OF THE INVENTION

An object of the invention is to provide an improved single lumen catheter for medical uses.

Another object of the invention is to provide a catheter tip that has the same OD as the catheter tube to aid in insertion by reducing the size of the tip.

Yet another object of the invention is to provide a tip that has a reinforcing arch or dimple that minimizes kinking.

Another object of the invention is to provide a tip that can be made economically.

Yet another object of the invention is to provide a tip that will not separate from the tube.

Yet another object of the invention is to provide a tip that is as strong as the tube itself.

Yet another object of the invention is to provide a tip:
that can be inserted with no internal stiffening system or utilizing a stylet, over a guide wire or through a sheath.
that aids in preventing occlusion in the aspiration mode
that effectively slows flow and diffuses the infusate as it exists the tip port without slowing the effective infusion rate through the catheter
that eliminates the needs for side holes
that does not injure the vessel wall during insertion or while resting in situ
that reduces the possibility of fibrin sheath build up.

The foregoing and other objects are realized by first skiving or removing a portion of an end of a single lumen tube. The tube end is then inserted into a mold and molten polyurethane or another thermoplastic material or thermoset material such as silicons is injected into the tool to form the tip. A reinforcing arch or dimple is formed when the heat and pressure of the molten plastic deforms the floor of the skived tube portion to permanently form the arch. The mold itself has an open space between the tube wall and the wall of the mold cavity. The floor of the tube is forced into this cavity to form the arch.

### BRIEF DESCRIPTION OF THE DRAWINGS

The invention, including its construction and method of construction is illustrated more or less diagrammatically in the drawings in which:

FIG. 1 is a side elevational view of a portion of a medical catheter embodying features of the invention.

FIG. 2 is a top plan view of the bolus of the catheter shown in FIG. 1.

FIG. 3 is bottom plan view of the bolus of the catheter shown. in FIG. 1.

FIG. 4 is an angled plan view of the top and leading end of the catheter tip shown in FIG. 1.

FIG. 5 is a longitudinal sectional view taken through the bolus of the catheter Bolus of the catheter of FIG. 1 showing the deformed tube wall forming the reinforcing arch and the molded tip.

FIG. 6 is a side elevational view of the skived tubing portion of the catheter shown in FIGS. 1 and 5.

FIG. 7 is an angle elevational view of the skived tubing portion shown in FIG. 6.

FIG. 8 is a longitudinal sectional view taken through the skived tube shown in FIG. 6 showing the tube wall of the skived portion before it is deformed.

FIG. 9 is longitudinal sectional view of the tip and skived, but not deformed tube wall resting in a side elevational view of one half of the injection molding tool and the tool's gate for the infusion of molted plastic.

FIG. 10 is a longitudinal sectional view of the molded tip with the lower skived tube Wall deformed into the cavity in the base of the molding tool.

FIG. 11 is a side elevational view of the catheter as shown in FIG. 11.

FIG. 12 is a cross sectional view taken along line 12-12 of FIG. 11.

FIG. 13 is a cross sectional view taken along line 13-13 of FIG. 11.

FIG. 14 is a cross sectional view taken along line 14-14 of FIG. 11.

FIG. 15 is a cross sectional view taken along line 16-15 of FIG. 11.

FIG. 16 is a cross sectional view taken along line 16-16 of FIG. 11.

FIG. 17 is a longitudinal sectional view taken through the bolus of the catheter Bolus of FIG. 1 showing the correct alignment of 19, 17 and 25.

FIG. 18 is a longitudinal sectional view taken through the bolus of the catheter Bolus of FIG. 1 showing an incorrect alignment of 19, 17 and 25 whereby outflow is restricted.

FIG. 19 is a longitudinal sectional view taken through the bolus of the catheter Bolus of FIG. 1 showing an incorrect alignment of 19, 17 and 25 whereby the effectiveness of the dimple in preventing undo flexing and kinking is reduced.

FIG. 20 is a side elevational view of another embodiment of the catheter bolus.

FIG. 21 is a longitudinal sectional view through the bolus of FIG. 20.

### DETAILED DESCRIPTION OF THE PREFERRED EMBODIMENTS

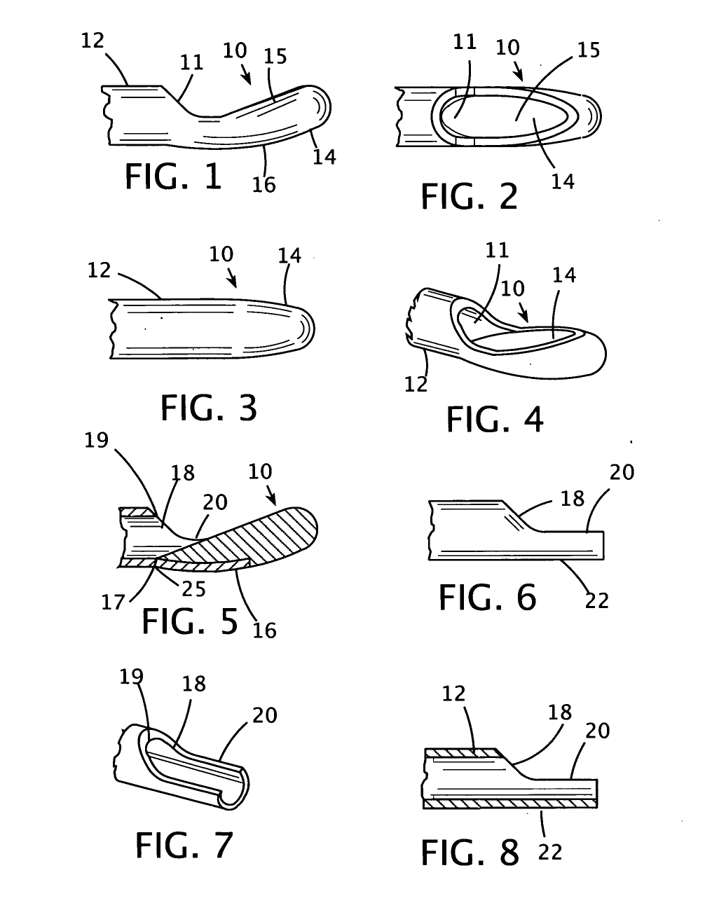

Referring now to drawing FIGS. 1-4, a single lumen catheter embodying features of the invention is shown generally at 10. The single lumen catheter shown at 10 comprises a catheter tube 12 onto which a polyurethane bolus tip 14 is insert molded. The reinforcing arch is shown at 16. Flow port 11 directs flow to directional ramp 15.

Now referring to drawing FIGS. 5-8. FIG. 5 shows the 45 degree downward skive forming the first portion of the port at 18. The flat skived portion of the tube forming the side of the port is shown at 20. The deformed tube portion 16 forming the arch is shown fused to the bolus 14. The undistorted lower tube wall is shown at 22.

The leading top edge 19 of port 11 is at the same cross sectional point as the point 17 where the ramp 15 meets the inner lumen wall 22 of tube 12. The deformation of the tube wall 22 begins at point 25 that is located at the same cross-sectional point as points 19 and 17. This positioning of all three points is important because it maintains full unrestricted outflow and maximum reinforcement by dimple 16. The position of port leading edge 19 and ramp 15 junction assures that the port opening is fully open for flow and is not restricted by a ramp/wall junction that would be inside the port. The port 11 resists kinking because it is reinforced by the 45 degree skive 18 and because the ramp 15 provides a thicking at the cross sectional point of the top of port 19 and the ramp 15 junction with tube wall 22.

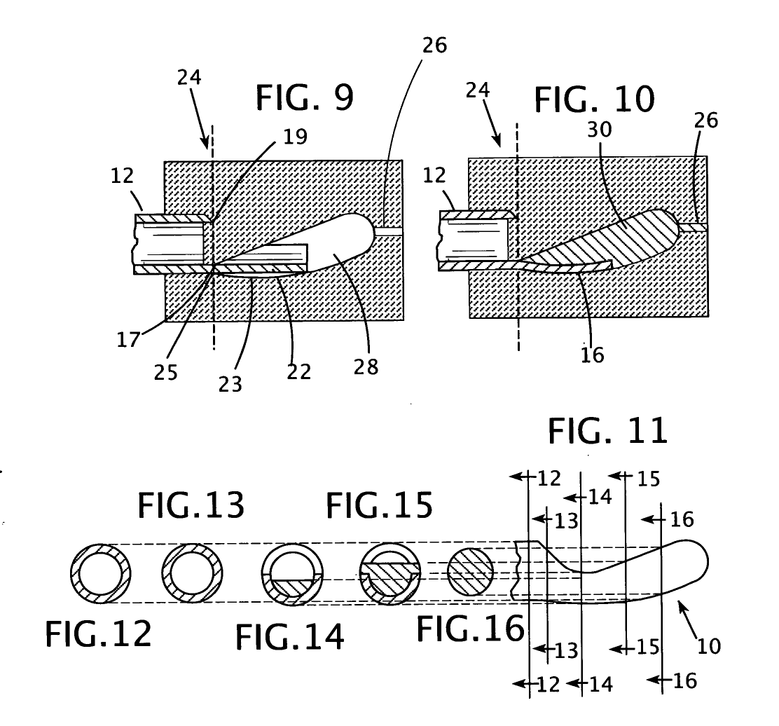

Referring to FIG. 9, one half of the main tool cavity is shown at 24. In FIG. 9 the gate 26 allows for the injection of molten polyurethane in to mold cavity 28. Skived tube 12 is placed in the cavity half and a mirror tool half closes to encase skived tube 12. Tube wall 22 is placed so that its leading edge 21 is in contact with the lower wall of cavity 28.

Referring to FIG. 10, molten polyurethane 30 enters tool cavity 28 and flows over the top of skived wall 22 turning it into reinforced arch 16 as the wall is forced down into tool cavity portion 23. The tube 12 is fused to the bolus portion 14,

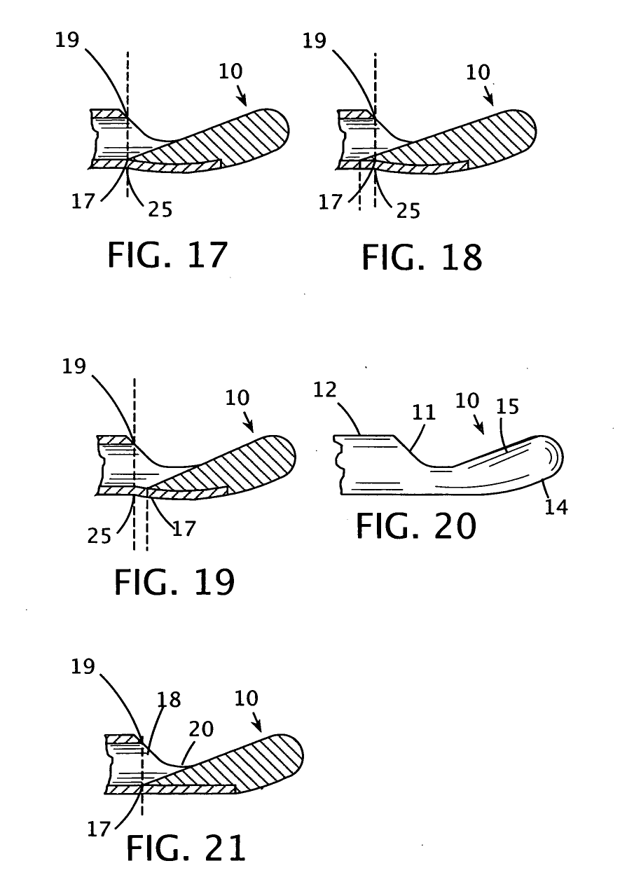

FIGS. 12-16 are cross-sectional views of FIG. 11. FIG. 11 is a side elevational view of the catheter. FIGS. 17-19 are longitudinal sectional views through the bolus which showing correct and incorrect alignments of points 17, 19 and 25.

FIG. 20 is a side elevational view of a portion of the previously described medical catheter shown in FIG. 1 with a reinforcing arch removed. The top point edge 19 and the point 17 where the ramp 15 meets the inner lumen wall 22 of the tube 12 are still at the same cross-sectional point and provide some anti-kink qualities.

1. A medical catheter, comprising:
a) a catheter tube having a proximal end and a distal end and including a single lumen inside a generally cylindrical side wall;
b) a segment of said side wall adjacent said distal end being removed so that an opening exists in said tube at said distal end and extending away from said distal end for a predetermined distance on one side of said tube;
c) a bolus molded onto the distal end of said tube and forming a tip on said catheter;
d) said bolus tip including a nose section extending forwardly of said distal end, and a connector section joining said bolus tip to, said tube in said opening and forming, with said opening a port in said one side of said tube.

2. The medical catheter of claim 1 further characterized by and including:
a) a radially outwardly extending stiffening arch formed in said catheter tube opposite said port.

3. A method of fabricating a medical catheter, comprising the steps of:
a) providing catheter tube having a proximal end and a distal end and including a singe lumen inside a generally cylindrical sidewall;
b) forming the distal end of said single lumen tube so as to create an opening at said distal end and extending away from said distal end for a predetermined distance on one side of said tube;
c) injection molding a bolus onto the distal end of said tube to form a tip on said catheter and create a port in one side of said catheter;
d) radially expanding said sidewall opposite said port during the molding process to form a stiffening arch in said catheter opposite said port.
